(19) United States
(12) Patent Application Publication
(10) Pub. No.: US 2018/0196924 A1
(43) Pub. Date: Jul. 12, 2018

(54) COMPUTER-IMPLEMENTED METHOD AND SYSTEM FOR DIAGNOSIS OF BIOLOGICAL CONDITIONS OF A PATIENT

(71) Applicant: International Business Machines Corporation, Armonk, NY (US)

(72) Inventors: Solomon Assefa, Ossining, NY (US); Geoffrey H. Siwo, Sandton (ZA); Gustavo A. Stolovitzky, Riverdale, NY (US)

(21) Appl. No.: 15/401,115

(22) Filed: Jan. 9, 2017

(51) Int. Cl. G06F 19/00; G06N 7/00; A61B 5/00

(52) U.S. Cl. CPC G06F 19/345; G06N 7/005; A61B 5/0022; A61B 5/7275; A61B 5/7282; A61B 5/7246

(57) ABSTRACT

A computer-implemented method of diagnosis of a patient comprises comparing a marker-print of a patient, wherein the marker-print comprises an N-value vector with each value in the vector indicative of a state of a biological marker of the patient, against a compendium of reference marker-prints, each reference marker-print having an associated biological condition, the reference marker-prints being stored in a marker-print database, to determine at least one reference marker-print having at least one matching value with the patient marker print. The method may comprise calculating, by a confidence module of the computer processor, a level of similarity between the patient marker-print and the at least one determined reference marker-print with the at least one matching value, thereby to provide an indication of a confidence level that the patient has the biological condition associated with the at least one determined reference marker-print having the at least one matching value.

### BACKGROUND

The present invention relates to diagnosis of biological conditions and it relates specifically to a computer-implemented method for enhancing biomarker-based diagnostics with prior knowledge of biological states.

### SUMMARY

According to an embodiment of the present invention, there is provided a method comprising providing a marker-print of a patient, wherein the marker-print comprises an N-value vector with each value in the vector indicative of a state of a biological marker of the patient. The method may comprise comparing, by a comparison module of a computer processor, the patient marker-print against a compendium of reference marker-prints, each reference marker-print having an associated biological condition as a label, the reference marker-prints being stored in a marker-print database, to determine at least one reference marker-print having at least one matching value with the patient marker print. The method may comprise calculating, by a confidence module of the computer processor, a level of similarity between the patient marker-print and the at least one determined reference marker-print with the at least one matching value, thereby to provide an indication of a confidence level that the patient has the biological condition associated with the at least one determined reference marker-print having the at least one matching value.

Embodiments of the present invention extend to a corresponding system and a computer program product.

According to another embodiment of the present invention, there is provided a computer-implemented method of enhancing a diagnosis of a patient. The method may comprise providing a marker-print of a patient and an associated primary diagnosis, wherein the marker-print comprises an N-value vector with each value in the vector indicative of a state of a biological marker of the patient. The method may comprise comparing, by a comparison module of a computer processor, the patient marker-print against a compendium of reference marker-prints, each reference marker-print having an associated biological condition, the reference marker-prints being stored in a marker-print database, to determine at least one reference marker-print having at least one matching value with the patient marker print. The method may comprise calculating, by a confidence module of the computer processor, a level of similarity between the patient marker-print and the at least one determined reference marker-print with the at least one matching value, thereby to provide an indication of a confidence level that the patient has the biological condition associated with the at least one determined reference marker-print having the at least one matching value, wherein, if the biological condition associated with the at least one determined reference marker-print matches the primary diagnoses, then the primary diagnosis is confirmed and wherein, if the biological condition associated with the at least one determined reference marker-print does not match the primary diagnoses, providing an enhanced diagnosis of a secondary biological condition

### BRIEF DESCRIPTION OF THE DRAWINGS

FIG. 1 illustrates a network topology comprising a computer system for diagnosis of a patient, in accordance with an embodiment of the invention;

FIG. 2 illustrates a schematic view of the computer system of FIG. 1 in more detail;

FIG. 3 illustrates a flow diagram of a method of diagnosis of biological conditions of a patient, in accordance with an embodiment of the invention;

FIG. 4 illustrates a flow diagram of a method of structuring data for use in the method of FIG. 3; and

FIG. 5 illustrates an example chart showing the operation of the computer system of FIG. 2 and the method of FIG. 3.

### DETAILED DESCRIPTION

The Applicant has observed that biological conditions may have a plurality of biological markers associated therewith. In the context of this specification, the term “biological marker” encompasses one or more measurable biological entities such as expression level of RNA transcripts, genotypes, epigenetic state, level or state of a protein/enzyme/metabolite/cell type, microbiome or any biomolecule as well as physiological and clinical markers such as heart rate, blood pressure, etc.

An embodiment of the present invention may solve the problem of providing accurate primary and/or secondary diagnoses of diseases or biological states/conditions using existing diagnostics and a compendium of known biological states by matching patterns of a plurality of biological markers (referred to as a “marker-print”) in a patient to those of biological states in a compendium. In the context of this specification, the term “biological states” may encompass labels or categories referring to conditions of biological samples such as disease names, types of cells/tissues/organs, chemical exposure, ancestry or differentiation/activation state of cells (e.g. activated T-cell), treatment state or outcomes of a biological entity, etc. “A compendium of biological states” may encompass biological data based on one or more biological markers such as genes, genotypes, epigenetic profiles or levels of metabolites/proteins/enzymes/cell types and any biomolecules and the associated biological states or tissues in a group of patients.

Most diagnostics are tested during clinical trials for use for very specific diseases or biological conditions and are later approved only for those conditions for which they were tested. In the context of this specification, the term “diagnostic” may encompass methods, equipment or tools used to infer state of health or disease or response to a biological intervention (e.g. drug or vaccine) or used to classify biological material into one or more groups. Yet, many biological conditions may be closely related and can therefore be diagnosed using the same set of biomarkers applied in different combinations. Thus, many diagnostics potentially can provide more clinical information than was originally intended and can be repurposed to assess additional diseases or provide secondary diagnoses beyond those they were originally intended.

An embodiment of the present invention provides methods for enhancing the results of diagnostic tests that rely on a set of a biological (clinical or genomics) markers, by matching the results of the diagnostic tests to a compendium of biological states for which the presence or absence of the set of markers is known. An embodiment of the present invention enhances a diagnostic test by one or more ways including identifying secondary diagnoses, minimizing misdiagnoses, increasing accuracy of diagnoses, refining diagnoses and leveraging enhanced diagnoses for therapy or prognosis when the biological state of interest is disease. “State of a biological marker” represents attributes of a biomarker such as gene activity (e.g. up or down or using continuous attributes), protein activity, enzyme activity, DNA methylation state, histone modification state, protein modification state, etc.

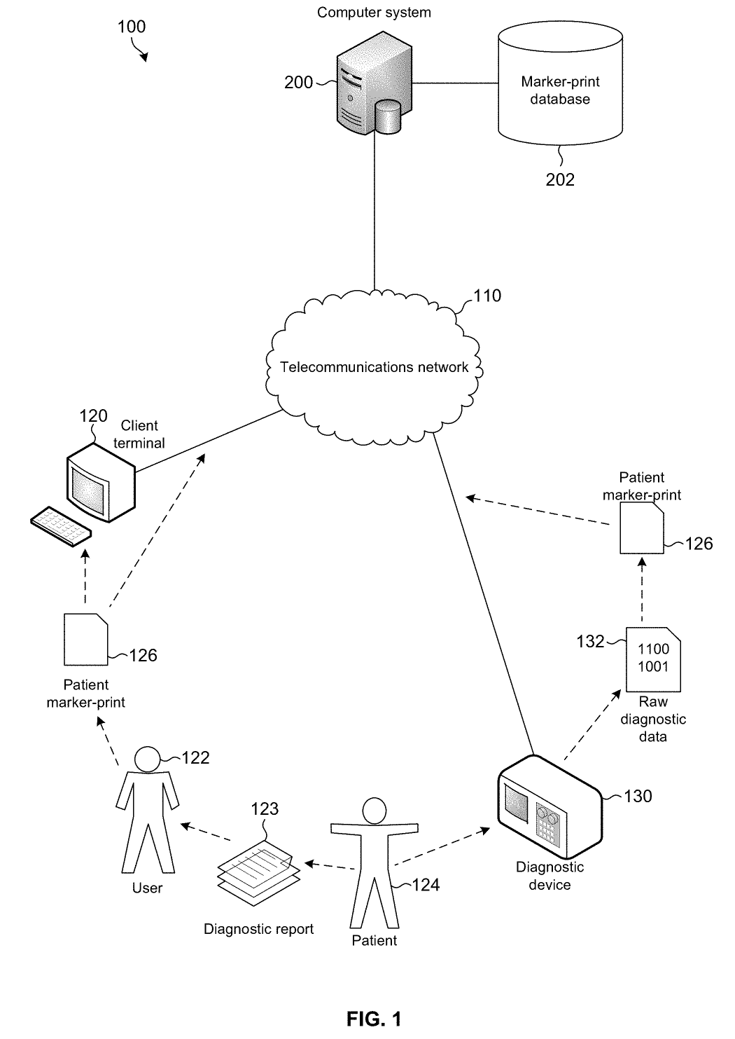

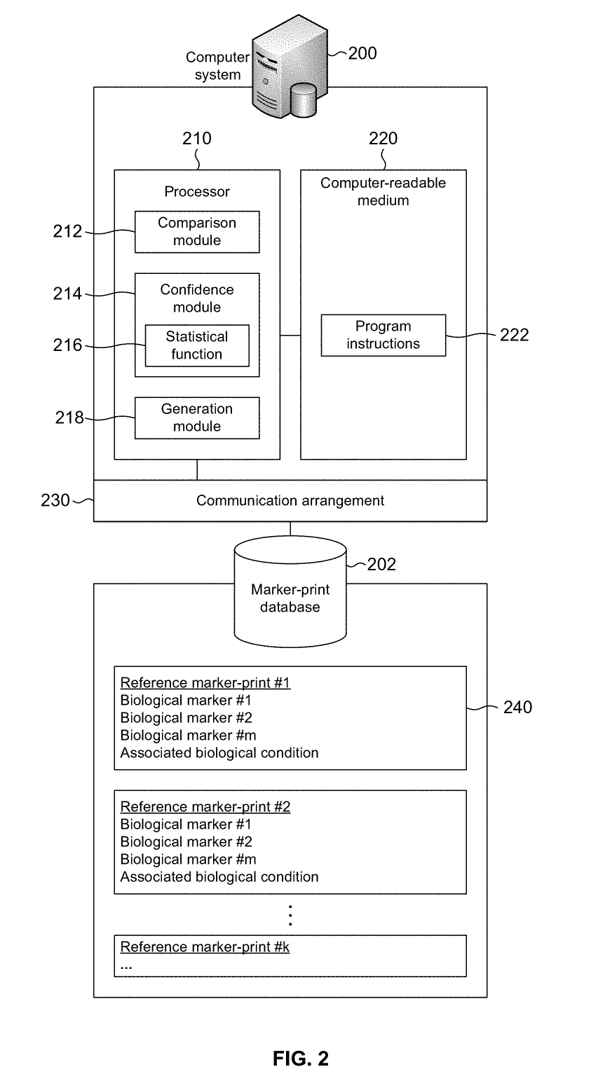

With reference now to FIG. 1, a network topology 100 includes a computer system 200 for diagnosis of biological conditions of a patient, in accordance with an embodiment of the invention. The computer system 200 is described in more detail (below) with reference to FIG. 2 and comprises (either integral therewith or separate and networked thereto) a marker-print database 202.

The computer system 200 may be communicatively coupled to a telecommunications network 110 which may be, or at least include, the internet. Accordingly, the computer system 200 may be connectable to remote computer and diagnostic devices which are also coupled to the telecommunications network 110. For example, a client terminal 120 may connect to, and access, the computer system 200 via the telecommunications network 110. The client terminal 120 may be a computer (e.g., desktop, laptop, tablet, mobile phone, etc.) at a medical lab. The client terminal 120 could be a medical diagnostic device which has network capabilities (e.g., a “smart” medical device) which can connect to a network using a built-in communication interface.

A patient 124 to be diagnosed need not interface directly with the computer system 200 (and need not even be aware of the computer system 200). A user 122 (e.g., a medical practitioner or lab technician) may deal with the patient 124 and be a human interface (if required) between the patient 124 and the computer system 200. The user 122 may also operate the client terminal 120, e.g., to input information or to retrieve information.

In order to diagnose the patient 124, using the system 200 and method in accordance with this embodiment of the invention, diagnostic data is required. The diagnostic data may be obtained from a conventional diagnostic test with a corresponding diagnostic report 123, of which there are many examples (e.g., blood tests, diagnostic device results, clinical evaluation, etc.). Results of previous diagnostic tests may be used. The diagnostic report 123 may be summarized or otherwise rendered into a format compatible with the computer system 200, which is an N-value vector. Where diagnostic results of the patient 124 have been formulated in the N-value vector (where N is greater than 1), it is referred to as a patient marker-print 126. A vector may be considered as a matrix with a single row or column. In a different embodiment, the marker-print may contain an N×M matrix. In this example embodiment, each of the N values of the vector relates to a single biological marker, and indicates a presence (or absence) of the biological marker.

The diagnostic report 123 may be manually converted, e.g., by the user 122, into a patient marker-print 126, for example using a data capture user interface provided by the client terminal 120. Instead, where diagnostic data 132 is obtained from an electronic diagnostic device 130, the diagnostic device 130 may be configured to render its raw diagnostic data 132 additionally into the patient marker-print 126. The diagnostic device 130 may include a communication interface (e.g., a network port or device) and may thus communicate directly with the computer system 200 with or without input required from the user 122.

Table 1 shows a first example of the patient marker-print 126.

TABLE 1

| | |
|---|---|
| M1 | + |
| M2 | + |
| M3 | − |
| M4 | + |

Table 2 shows a second example of the patient marker-print 126.

TABLE 2
M1
M2
M4

In Table 1, M1 . . . M4 refer to biological markers, while the sign (+ or −) indicates whether or not the biological marker is present. Table 2 indicates similar information but more concisely. Only biological markers which are present (M1, M2, and M4) are indicated in the table. Tables 1 and 2 convey similar information but illustrate that the marker-print (e.g., the patient marker-print 126) may take different forms.

FIG. 2 illustrates components of the computer system 200 in more detail. The computer system 200 comprises a computer processor 210 communicatively coupled to a computer-readable medium 220. The computer processor 210 may be one or more microprocessors, controllers, or any other suitable computing resource, hardware, software, or embedded logic. Program instructions 222 are stored on the computer-readable medium 220 and are configured to direct the operation of the processor 210. The processor 210 (under the direction of the program instructions 222) comprises a plurality of conceptual modules 212, 214, 218 which may correspond to functional tasks performed by the processor 210.

The marker-print database 202 has a plurality of reference marker-prints 240 stored thereon. The reference marker-prints 240 are also in the format of a vector, but may be an M-value vector, where M is not necessarily equal to N. The reference-marker prints 240 may be generated from historical diagnosis data where various confirmed biological markers have been associated with a biological condition (e.g. colon cancer). The reference marker-prints 240 may exclude any personally identifying information. There may be plural, even numerous, reference marker-prints 240 relating to the same biological condition, and these may have identical or overlapping biological markers.

A comparison module 212 is configured to compare the patient marker-print 126 to reference marker-prints 240 stored in the marker-print database 202. The comparison module 212 may implement a known matching algorithm to find one or more reference marker-prints 240 with at least one biological marker in common with the patient marker-print 126. The comparison module 212 may simply return reference marker-prints 240 which match, or may indicate quantitatively the number of matching biological conditions between the patient marker-print 126 and the reference marker-print(s) 240.

A confidence module 214 implements a statistical function 216 to provide an indication of a degree of matching, or a confidence level of matching, between the patient marker-print 126 and the one or more matching reference marker-prints 240. A degree of matching may be provided by a confidence value, which may be generated in one or more ways including but not limited to the use of a hypergeometric test derived P-value incorporating the number of elements in the patient marker-print 126, the number of elements in the reference marker-print 240 in the marker-print database 202 and a total number of unique elements in the marker-print database 202. The confidence value may also be generated using the absolute count of the number of elements in the patient marker-print 126 that exactly match the elements in the reference marker-print 240 in the database 202.

Alternatively, the proportion of elements in the patient marker-print 126 that exactly match the elements in the reference marker-print 240 in the database 202 can be used to generate a score. Alternatively, the confidence score may be generated by estimating the likelihood of observing a specific fraction of elements in the patient marker-print 126 in randomly generated vectors of the same number of elements of the patient marker-prints, whereby each random vector is generated by randomly sampling elements from a vector of all unique elements in the marker-print database 202. When the patient marker-print 126 and reference marker-print 240 both consist of continuous values, the confidence value is generated using statistical procedures for assessing similarity between vectors including but not limited to Spearman or Pearson correlation coefficient, or cosine similarity.

A generation module 218 is not necessarily required for matching and diagnosing, but rather for automated generation of marker-prints. The generation module 218 is configured to interrogate or scan diagnostic data, e.g., diagnostic reports, data output from diagnostic devices, user input, or the like, and to render the information in marker-print form (e.g., an N-value vector). The generation module 218 may find application prior to matching. The generation module 218 may be applied to patient data to generate the patient marker-print 126 and/or to reference data in order to generate the reference marker-print 240.

The marker-print database 202 has stored thereon a plurality of reference marker-prints 240, each including a plurality of biological markers as well as an associated biological condition (or biological signature). Each reference marker-print 240 may be stored as a separate record in the marker-print database 202. The marker-print database 202 may be continually updated as more reference data, and associated reference marker-prints 240, are generated. The marker-print database 202 and/or computer system 200 may be configured to comply with relevant data protection/personal information/medical information laws and regulations in the region(s) it which they are operated.

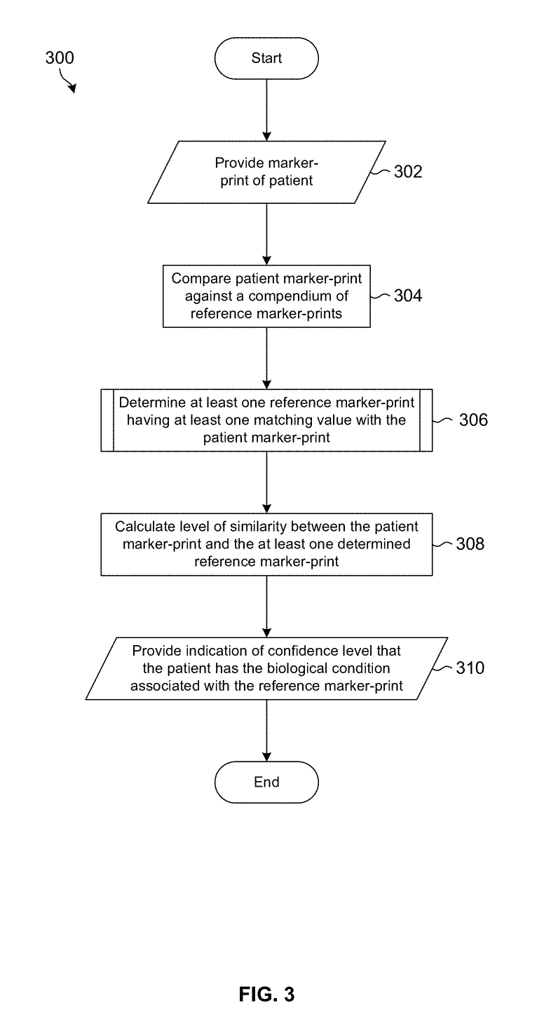

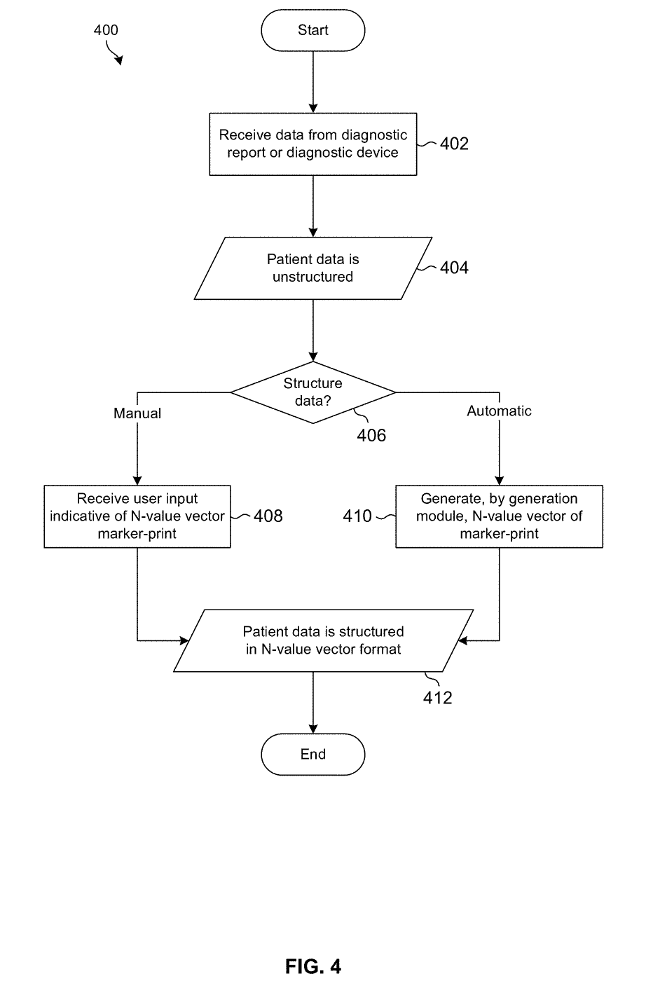

An embodiment of the invention will now be further described in use, with reference to FIGS. 3-4.

FIG. 3 illustrates a flow diagram of a method 300 of diagnosis of biological conditions of a patient, in accordance with an embodiment of the invention. The method 300 may be implemented by the computer system 200; however, it is understood that the method 300 may be implemented by a different computer system and that the computer system 200 may be configured to implement a different method.

The patient marker-print 126 is provided (at block 302). The patient marker-print 302 may be provided in more than one way and two optional ways are illustrated in FIG. 4 (further described below). Regardless of how the patient marker-print 126 is generated, it is in N-value vector format with suitable listed and formatted indicators of N biological conditions. The provision of the patient marker-print 126 may include communicating the patient marker-print 126 to the computer system 200 from a remote location, e.g., the client terminal 120 or the connected diagnostic device 130.

The comparison module 212 compares (at block 304) the provided patient marker-print 126 against the compendium of reference marker-prints 240 in the marker-print database 202. The comparison module 212 determines (at block 306) at least one reference marker-print 240 having at least one biological indicator in common with the patient marker-print 126. The comparison module 212 may be configured to include basic filter criteria, e.g., only determine the a reference marker-print 240 has more than a certain number (e.g., two) of matching biological markers or more than a certain percentage, e.g., 50%. However, in this example embodiment, any filtering or ranking is provided by the confidence module 214.

The confidence module 214 is configured to calculate (at block 308) a level of similarity between the patient marker-print 126 and the determined reference marker-print(s) 240. This provides an indication of the likeliness or “confidence” that the patient 124 has the biological condition(s) associated with the matching reference marker-print(s) 240. The criteria on which the confidence module 214 is configured to base the level of similarity may include:
a number of biological conditions listed in the patient marker-print 126 (e.g., the value of N);
a number of biological conditions listed in the reference marker-print 126 (e.g., the value of M);
a number of matching biological conditions matched between the patient marker-print 126 and the reference marker-print 240;
a number of reference marker-prints 240 having the same biological conditions which match the patient marker-print 126;
a sample size of the reference marker-print 240;
or the like.

The confidence module 214 configured to provide (at block 310) a quantitative or qualitative probability, based on the available information by implementing the statistical function 216, that the patient 124 has the biological condition associated with the matching (or partially matching) reference marker-prints 240. An output indicative of the results of the comparison module 212 and confidence module 214 determinations may be saved (e.g., on the marker-print database 202) and/or communicated to one or more recipients. The output may be formulated as a computerized diagnosis and communicated to the patient 124, the user 122, and/or other interested and affected parties.

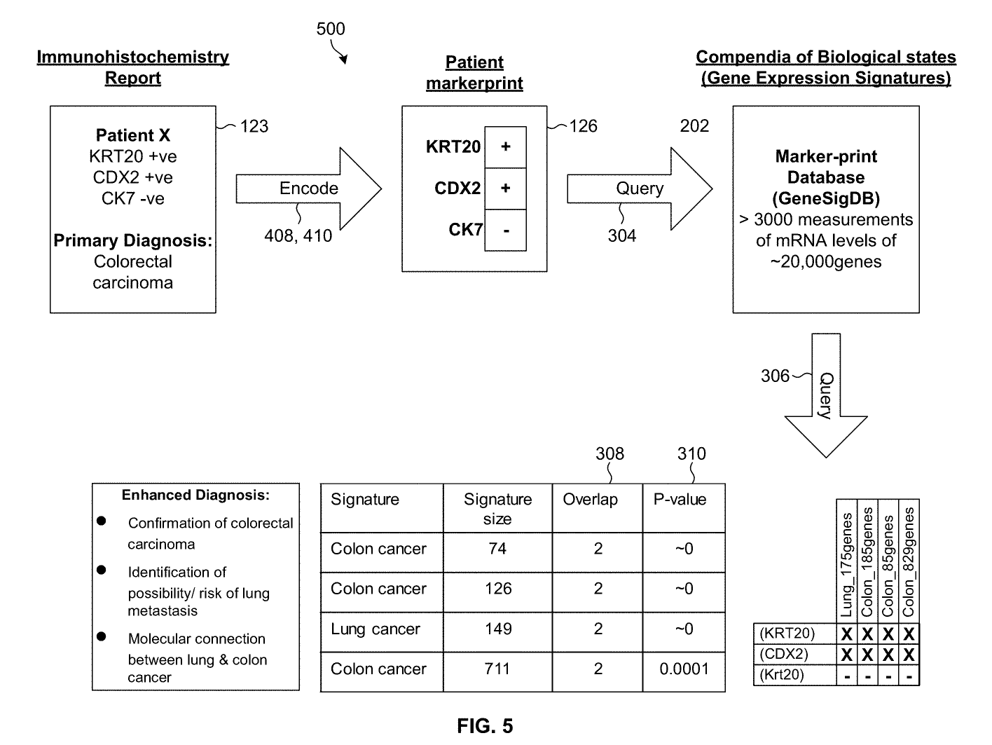

There may be plural uses for this computerized diagnosis. For example:
The computerized diagnosis may be used for the purposes of at least one secondary diagnosis to the patient 124 based on similar patterns of biological markers in the patient marker-print 126 and those of the reference marker-print 240. In such case, the method 300 may be a computerized method of providing a secondary diagnosis.
The computerized diagnosis may be used for the purposes of refining a primary diagnosis from a marker based test. For example, reducing false positive results or reducing misdiagnoses from a marker based test by identifying other biological states with reference marker-prints 240. In such case, the method 300 may be a computerized method of reefing a diagnosis.
The computerized diagnosis may be used for the purposes of leveraging the secondary and/or refined diagnoses to select therapeutics or predict disease prognosis in a patient. For example, an implied molecular connection between lung and colon cancer points to the possibility of using therapeutics for one cancer for the other (refer to FIG. 5).
The computerized diagnosis may be used for the purposes of matching a set of biological markers in a diagnostic test result to a tissue or combination of tissues, cell states or cell types, for example, to detect tissue contamination or mixtures of tissues at a forensic site.
The computerized diagnosis may be used for the purposes of predicting progression of disease or biological states. For example, predicting possible tissues to which a patient-cancer may undergo metastasis based on the similarity between markers in the patient 124 and those in the compendium of biological states including other cancer types or tissues. In such case, the method 300 may be a computerized method for predicting progression of disease or biological states.
The computerized diagnosis may be used for repurposing a previous diagnostic test for one disease to detect another disease or condition for which it was not initially designed or approved by finding additional diseases with a similar marker-print in the biological compendium. In such case, the method 300 may be a computerized method of repurposing results of a previous diagnostic test.
The computerized diagnosis may be used for the purposes of in silico prediction of other diseases or biological states that may be diagnosed by a set of markers in an existing diagnostic test by generating in silico all possible combinations of marker-prints and matching them to the compendium of reference marker-prints from several biological states. In such case, the method 300 may be a computerized method of in silico prediction of diseases or biological states.
The computerized diagnosis may be used for the purposes of validation of a set of biological markers for a given disease state based on concordance between the patient marker-prints 126 generated from a diagnostic to those of a compendium of reference marker-prints 240 across diverse biological states. In such case, the method 300 may be a computerized method of validation of a set of biological markers. The method may include validating the ability of reference marker-prints 240 to predict a biological state using a distinct set of biological markers in a diagnostic, whereby the reference marker-prints 240 in the compendium may be of the same or different molecular type as those in the diagnostic. For example, the set of biological markers in the diagnostic may be immunohistochemical while the set of markers in the compendium may be RNA transcript levels of genes encoding the proteins detected by the immunohistochemical diagnostic.
The computerized diagnosis may be used for the purposes of matching all possible combinations of biological markers in a patient marker-print 126 in a diagnostic to the compendium of reference marker-prints 240 in various biological states to construct a database of marker combinations corresponding to any given biological state. The resulting database of marker combinations and their corresponding biological states may be much smaller than the original marker-print database 202 and may therefore be stored at lower computer memory for rapid and off-line matching of diagnostic results to diverse biological states/diseases. For example, to enable the methodology of an embodiment of this invention to be applied in resource limited situations with slow or no internet connectivity and/or in mobile devices.
The computerized diagnosis may be used for the purposes of identifying the minimum set of biological markers that if constituted in a marker-print 126 would diagnose the maximum possible number of diseases or biological conditions.
The computerized diagnosis may be used for the purposes of comparing patients and assigning them to groups based on their sets of marker-prints at a single point of time or longitudinally.

FIG. 4 illustrates a flow-diagram of an example method 400 for rendering or encoding a marker-print (whether a patient marker-print 126 or a reference marker-print 240). The method 400 comprises compiling or receiving (at block 402) diagnostic data from a diagnostic report 123 or a diagnostic device 130. The received data is unstructured (at block 404) in the sense that it is not yet in the format of an N-value array suitable for a marker-print. If the diagnostic data is from the diagnostic report, it may be in a human-readable format. If the diagnostic data is from the diagnostic device 130, it may be in a machine-readable format.

The method 400 comprises structuring (at block 406) the diagnostic data in one of two ways. In a manual process, the data is structured by the user 122 who enters the structured data via the client terminal 120. Accordingly, the method 400 comprises receiving (at block 408) a user input indicative of the N-value vector marker-print. In an automatic process, the N-value vector marker-print is generated (at block 410) programmatically by a computer, e.g., the computer system 200 or the diagnostic device 130. For example, the diagnostic device 130 may communicate the raw diagnostic data 132 to the computer system 200 for generating, by the generation module 218, the marker-print. In a different embodiment (not illustrated), the generation module may be provided in the diagnostic device 130 itself. The outcome may be data (e.g., patient data) structured (at block 412) in the N-value vector marker-print format.

FIG. 5 illustrates a chart 500 populated with example biological markers and biological conditions to illustrate the operation of the computer system 200 and method 300. An immunohistochemistry report (which is an example of a diagnostic report 123) is provided by the user 122, which may be a physician or clinician of the patient 126. The user 122, using the immunohistochemistry report 123, has already provided a primary diagnosis of the patient 126, the primary diagnosis being colorectal carcinoma.

The immunohistochemistry report 123 is encoded (at block 408, 410) into a patient marker-print 126. Line items of the N-value vector (where N=3, in this example) are representative of the biological conditions provided in the immunohistochemistry report 123. The patient marker-print 126 is communicated (e.g., via the client terminal 120) to the computer system 200. The comparison module 212 matches (at block 306) the patient marker-print 126 with reference marker-prints 240 in the marker-print database 202 hosting the compendium of marker-prints.

In the chart 500, there are four top matching reference marker-prints 240, each with an associated biological condition or signature. Both a similarity or overlap is calculated (at block 308) and a statistical significance is calculated (at block 310) by the confidence module 214. In this chart 500, the most statistically significant match is colon cancer, thus confirming the primary diagnosis. However, the match also indicates a possibility of lung cancer and possibly indicates a correlation between lung cancer and colon cancer.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method of diagnosis of a patient, the method comprising:
providing a marker-print of a patient, wherein the marker-print comprises an N-value vector with each value in the vector indicative of a state of a biological marker of the patient;
comparing, by a comparison module of a computer processor, the patient marker-print against a compendium of reference marker-prints, each reference marker-print having an associated biological condition, the reference marker-prints being stored in a marker-print database, to determine at least one reference marker-print having at least one matching value with the patient marker print; and
calculating, by a confidence module of the computer processor, a level of similarity between the patient marker-print and the at least one determined reference marker-print with the at least one matching value, thereby to provide an indication of a confidence level that the patient has the biological condition associated with the at least one determined reference marker-print having the at least one matching value.

2. The method of claim 1, further comprising a prior step of obtaining the marker-print of the patient which comprises at least one of:
receiving from a user, via a user interface, a user input indicative of the N-value vector; or
generating, by a generation module of the computer processor, the N-value vector based on raw diagnostic data captured by a diagnostic device.

3. The method of claim 1, wherein the N-value vector comprises one of:
an identifier of the biological condition together with a binary indication of whether or not the biological condition is present; or
an identifier of the biological condition only, wherein all identified biological conditions are present.

4. The method of claim 1, wherein the calculating, by the confidence module, comprises implementing a statistical function having, as an input, an indication of the at least one matching value and, as an output, the level of similarity between the patient marker-print and the at least one determined reference marker-print with at least one matching value.

5. The method of claim 4, which comprises defining a numerical confidence threshold against which the calculated level of similarity is compared to yield a likelihood of having, or not having, the biological condition.

6. The method of claim 1, which comprises determining, by the comparison module, a plurality of reference marker-prints having at least one matching value with the patient marker print.

7. The method of claim 6, which comprises calculating, by the confidence module, a level of similarity between the patient marker-print and each one of the plurality of determined reference marker-prints with at least one matching value.

8. The method of claim 1, further comprising a prior step of populating the marker-print database which comprises at least one of:
receiving from a user, via a user interface, a user input indicative of the reference marker-print in the form of an M-value vector and the associated biological condition; or
generating, by a generation module of the computer processor, the reference marker-print in the form of an M-value vector and the associated biological condition based on historical raw diagnostic data.

9. A computer system for diagnosis of biological conditions of a patient, the system comprising:
a computer processor;
a marker-print database comprising a compendium of reference marker-prints, each reference marker-print having an associated biological condition; and
a computer readable storage medium having stored thereon program instructions executable by the computer processor to direct the operation of the processor, wherein the computer processor, when executing the program instructions, comprises:
a comparison module configured to compare a marker-print of a patient, wherein the marker-print comprises an N-value vector with each value in the vector indicative of a biological marker of the patient, against the compendium of reference marker-prints, to determine at least one reference marker-print having at least one matching value with the patient marker print; and
a confidence module configured to calculate a level of similarity between the patient marker-print and the at least one determined reference marker-print with the at least one matching value, thereby to provide an indication of a confidence level that the patient has the biological condition associated with the at least one determined reference marker-print having the at least one matching value.

10. The computer system of claim 9, comprising a generation module configured to generate the N-value vector based on raw diagnostic data captured by a diagnostic device.

11. The computer system of claim 9, wherein the N-value vector comprises one of:
an identifier of the biological condition together with a binary indication of whether or not the biological condition is present; or
an identifier of the biological condition only, wherein all identified biological conditions are present.

12. The computer system of claim 9, wherein the confidence module is configured to implement a statistical function having, as an input, an indication of the at least one matching value and, as an output, the level of similarity between the patient marker-print and the at least one determined reference marker-print with at least one matching value.

13. The computer system of claim 12, wherein the confidence module comprises a numerical confidence threshold against which the calculated level of similarity is compared to yield a likelihood of having, or not having, the biological condition.

14. The computer system of claim 9, wherein the comparison module is configured to determine a plurality of reference marker-prints having at least one matching value with the patient marker print.

15. The computer system of claim 14, wherein the confidence module is configured to calculate a level of similarity between the patient marker-print and each one of the plurality of determined reference marker-prints with at least one matching value.

16. The computer system of claim 9, comprising a generation module which is configured to generate the reference marker-print in the form of an M-value vector and the associated biological condition based on historical raw diagnostic data.

17. A computer program product for diagnosis of biological conditions of a patient, the computer program product comprising:
a computer-readable medium having stored thereon:
a compendium of reference marker-prints, each reference marker-print having an associated biological condition, the reference marker-prints;
first program instructions executable by a computer processor to cause the computer processor to compare a patient marker-print against the compendium of the reference marker-prints, to determine at least one reference marker-print having at least one matching value with the patient marker print; and
second program instructions executable by the computer processor to cause the computer processor to calculate a level of similarity between the patient marker-print and the at least one determined reference marker-print with at least one matching value, thereby to provide an indication of a confidence level that the patient has the biological condition associated with the at least one determined reference marker-print having at least one matching value.

18. The method of claim 1, for enhancing the diagnosis of the patient, the method comprising providing, in addition to the marker-print of the patient, an associated primary diagnosis, wherein:
if the biological condition associated with the at least one determined reference marker-print, as determined by the confidence module, matches the primary diagnoses, then the primary diagnosis is confirmed; or
if the biological condition associated with the at least one determined reference marker-print, as determined by the confidence module, does not match the primary diagnoses, providing an enhanced diagnosis of a secondary biological condition.
